(19) United States
(12) Patent Application Publication
(10) Pub. No.: US 2007/0154498 A1
(43) Pub. Date: Jul. 5, 2007

(54) Intravenous essential fatty acid emulsion

(76) Inventors: Jonathan David Bortz, St. Louis, MO (US); R. Saul Levinson, Chesterfield, MO (US)

Correspondence Address: KENYON & KENYON LLP, ONE BROADWAY, NEW YORK, NY, 10004, US

(21) Appl. No.: 11/635,743

(22) Filed: Dec. 6, 2006

Related U.S. Application Data
(60) Provisional application No. 60/597,562, filed on Dec. 9, 2005.
(60) Provisional application No. 60/781,137, filed on Mar. 10, 2006.

(51) Int. Cl. A61K 38/18; A61K 38/43; A61K 31/714; A61K 31/59; A61K 31/525; A61K 31/51; A61K 31/401; A61K 31/22; A61K 31/366; A61K 38/29

(52) U.S. Cl. 424/400; 424/423; 514/12; 514/547; 514/167; 514/458; 514/763; 514/52; 514/251; 514/276; 514/350; 514/356; 514/423; 514/548; 514/460; 424/94.1; 424/646

(57) ABSTRACT

A method for preventing stenosis and thrombosis of an AV graft is disclosed. An essential fatty acid emulsion is administered to the patient through the AV graft, preferably during dialysis, whereby the anti-inflammatory properties of the essential fatty acid emulsion prevent complications typical of AV grafts.

### SCOPE OF THE INVENTION

The present invention relates to compositions including essential fatty acids suitable for intravenous administration to a patient in need thereof to reduce or eliminate inflammatory responses, as well as methods of making and using the same. More specifically, the present invention relates to compositions including an essential fatty acid emulsion suitable for intravenous use prior to or during hemodialysis to prevent or reduce stenosis and/or thrombosis of a vascular access.

### BACKGROUND OF INVENTION

Hemodialysis is the most common method used to treat advanced and permanent kidney failure. Since the 1960's, when hemodialysis first became a practical treatment for kidney failure, many advances have been made to make hemodialysis treatments more effective and to minimize side effects. During hemodialysis, a patient's blood is allowed to flow through tubing, a few ounces at a time, into a hemodialysis machine. The hemodialysis machine has three primary functions that include pumping blood and monitoring blood flow, cleaning waste from the blood and monitoring blood pressure and the rate of fluid removal from the blood. After passing through the hemodialysis machine, the cleaned blood is returned to the patient's body via tubing.

Before hemodialysis is performed, a vascular access, or site from which the blood is removed and returned must be prepared on the patient's body. A vascular access is typically prepared weeks to months before beginning hemodialysis. The vascular access needs to be capable of supporting a blood flow of approximately 250 milliliters per minute (ml/min).

Two common types of vascular access suitable for hemodialysis are the arteriovenous graft (AV graft) and the arteriovenous fistula (AV fistula.) An AV graft is a vascular access that uses a synthetic tube implanted under the skin typically in the patient's arm. One end of the implanted tube is attached to an artery and the other end of the tube is attached to a vein. The tube serves as an artificial vein that can be used repeatedly for needle placement and blood access during hemodialysis. An AV graft can be used for hemodialysis within about two weeks of implantation. Unfortunately, a high percentage of AV grafts develop low or inadequate blood flow due to stenosis or thrombosis within weeks or months of implantation. Low or inadequate blood flow is an indication of clotting or narrowing of the vascular access. In this case, a surgical procedure, such as angioplasty to widen the segment that has become narrowed, is required to reestablish a higher or more adequate blood flow for purposes of hemodialysis. An alternative option in the case of low or inadequate blood flow, is to perform surgery on the AV graft and replace the narrowed segment.

Up to 75% of AV grafts fail within 2 years of implantation, and some AV grafts require revision or declotting up to 4 times per year. Antiplatelet or anticoagulation regimens used in an attempt to reduce the AV graft failure rate have met with mixed results. The unwanted side effects of antiplatelet or anticoagulation regimens have all but precluded such approaches to reduce the high incidence of vascular access failures.

AV fistulas are less likely than AV grafts to form clots or become infected, and tend to last longer than any other type of vascular access. An AV fistula vascular access is formed by surgically connecting an artery directly to a vein, usually in the forearm. Directly connecting an artery to a vein causes more blood to flow into the vein. As a result, the vein grows larger and stronger, making repeated needle punctures for hemodialysis treatments easier. Unfortunately, AV fistulas, likewise have drawbacks. One such drawback is that it takes time for the vein to grow larger to create a robust and enlarged ‘rapidly flowing lake’ of blood for purposes of hemodialysis. At a minimum, 6 to 12 months are required for the vein to mature for hemodialysis use. Sometimes, as long as 24 months is required for maturation of the AV fistula for hemodialysis use.

Complications can arise with both AV grafts and AV fistulas that may require further treatment or surgery. The most common complications are infection and low blood flow due to blood clotting. Compared with AV fistulas, AV grafts tend to have more complications associated with clotting or infection requiring replacement of the AV graft. There is therefore a need to reduce or eliminate thrombosis and stenosis induced AV graft failure.

### SUMMARY OF INVENTION

The present invention provides compositions including an effective amount of essential fatty acids (EFAs) suitable for intravenous use in patients prior to or during hemodialysis to reduce or eliminate the incidence of vascular access stenosis and/or thrombosis.

The present invention also provides methods of reducing or eliminating the incidence of vascular access stenosis and/or thrombosis by intravenously administering to a patient in need thereof compositions including an effective amount of EFAs for reducing or eliminating stenosis and/or thrombosis.

The present invention further provides a method of reducing or eliminating the incidence of stenosis and/or thrombosis of a hemodialysis patient's vascular access. The method comprises administering intravenously a suitable composition including and effective amount of EFAs directly through a patient's vascular access.

The present invention further provides methods of manufacturing compositions including and effective amount of EFAs suitable for intravenous use in patients.

Accordingly, it is an object of the present invention to provide compositions suitable for intravenous use effective in the prevention, stabilization, reversal and/or treatment of vascular graft stenosis and/or thrombosis.

Another object of the present invention is to provide safe compositions suitable for intravenous use for the prevention, stabilization, reversal and/or treatment of vascular graft stenosis and/or thrombosis.

Another object of the present invention is to provide an effective method of preventing, stabilizing, reversing and/or treating vascular graft stenosis and/or thrombosis prior to or during hemodialysis.

Another object of the present invention is to provide a safe method of preventing, stabilizing, reversing and/or treating one or more complications associated with vascular grafts.

Another object of the present invention is to provide a method of manufacturing safe compositions suitable for intravenous use for the prevention, stabilization, reversal and/or treatment of one or more complications associated with vascular grafts.

Still another object of the present invention is to provide a method of manufacturing compositions including an effective amount of essential fatty acids suitable for intravenous use for the prevention, stabilization, reversal and/or treatment of one or more complications associated with vascular grafts useful for hemodialysis.

These and other objectives and advantages of the present invention, some of which are specifically described and others that are not, will become apparent from the detailed description and claims that follow.

### DETAILED DESCRIPTION

The present invention is directed to compositions containing an effective amount of essential fatty acids (EFAs) suitable for intravenous use to prevent, reverse, stabilize, reduce and/or eliminate one or more complications associated with vascular accesses such as stenosis and/or thrombosis. Compositions of the present invention are effective in preventing, reversing, stabilizing, reducing and/or eliminating one or more complications associated with vascular accesses by virtue of the anti-inflammatory and antithrombotic effects of the EFAs contained therein. Compositions of the present invention are particularly useful in cases wherein the vascular accesses are utilized for hemodialysis, although compositions of the present invention may be used with any intravenous access, whether for renal or non-renal patients. Compositions of the present invention are particularly useful in preventing, reversing, stabilizing, reducing and/or eliminating one or more complication associated with AV grafts and/or AV fistulas.

Preferred compositions of the present invention include one or more EFAs, or a fat emulsion containing one or more EFAs, such as one or more polyunsaturated, long-chain, omega-3 fatty acid containing 18 to 22 C atoms, omega-6 fatty acids, their pharmaceutically tolerable esters, their pharmaceutically tolerable salts or combinations thereof. Suitable EFAs may be utilized in their pure forms, or as components of oils, highly purified oil concentrates or linseed oil.

Additional EFA formulations include omega 3 and omega 6 fatty acids such

OMEGA 6 FAMILY

| Common Name | Numeric Name |
|---|---|
| Linoleic acid | 18:2n−6 |
| Gamma linolenic acid | 18:3n−6 |
| — | 20:2n−6 |
| Dihomo gamma linolenic acid (DHGLA) | 20:3n−6 |
| Arachidonic acid | 20:4n−6 |
| Docosatetraenoic acid | 22:4n−6 |
| — | 22:5n−6 |

OMEGA 3 FAMILY

| Common Name | Numeric Name |
|---|---|
| Alpha linolenic acid (ALA) | 18:3n−3 |
| Parinaric acid | 18:4n−3 |
| | 20:3n−3 |
| Eicosatetraenoic acid | 20:4n−3 |
| Eicosapentaenoic acid (EPA) | 20:5n−3 |
| Docosapentaenoic acid (DPA) | 22:5n3 |
| Docosahexaenoic acid (DHA) | 22:6n−3 |

and monounsaturated fatty acids such as:

| Common Name | Numeric Name |
|---|---|
| Myristoleic acid | 14:1 |
| none | 15:1 |
| Palmitoleic acid | 16:1 |
| none | 17:1 |
| Oleic Acid | 18:1 |
| Gadoleic acid | 20:1 |
| Erucic acid | 22:1 |
| Nervonic acid | 24:1 |

contain a single carbon-carbon double bond, whereas polyunsaturated fatty acids contain two or more double carbon bonds. These formulations of EFAs can also include mixtures of two or more fatty acids together such as: Gamma linolenic acid and and EPA and DHA etc. These various fatty acids can be produced synthetically or found in natural sources. For example, linoleic acid (LA) is found in commonly used cooking oils, including sunflower, safflower, corn, cottonseed, and soybean oils. Omega-6 fatty acids in the form of gamma linolenic acid (GLA) and LA are found in the plant seed oils of evening primrose, black currant, borage, and fungal oils.

Suitable omega-3 fatty acids include for example but are not limited to α-linolenic acid, eicosapentaenoic acid (EPA), and docosahexaenoic acid (DHA). Compositions of the present invention may include one or more suitable omega-3 fatty acids. The omega-3 fatty acids may be used in their pure form or in the form of components of fish oils. Suitable fish oils include those oils technically recovered in substantial quantities from cold-water fish, such as pilchard oil, menhaden oil, Peruvian fish oil, sardine oil, salmon oil, herring oil, and mackerel oil. Purified fish oil concentrations that are produced from mackerel, sardines, herrings, or salmon are preferred, wherein the EPA content of the oil concentration is 20 to 40%, and more preferably at least 26% by weight.

Suitable omega-6 fatty acids include for example but are not limited to linoleic acid, γ-linolenic acid, dihomo-γ-linolenic acid and arachidonic acid, whereby γ-linolenic acid and dihomo-γ-linolenic acid are preferred. Compositions of the present invention may include one or more suitable omega-6 fatty acids. The omega-6 fatty acids may be used in their pure form or in the form of components of oils, for example, primrose oil, borage oil or soybean oil, of which primrose oil is preferred.

Suitable pharmaceutically tolerable esters and salts of the noted omega-3 and omega-6 fatty acids may likewise be used in compositions of the present invention, whereby the pharmaceutically tolerable esters of these acids are particularly preferred. Pharmaceutically tolerable esters of the omega-3 and omega-6 fatty acids include for example but are not limited to the ethyl esters or glycerin esters, for example, mono-, di-, or triglycride esters, whereby triglycerides are preferred. Pharmaceutically tolerable salts of the omega-3 and omega-6 fatty acids include for example but are not limited to sodium salts thereof.

Compositions of the present invention comprise EFAs and/or a fat emulsion of EFAs including a mixture of fish oil and/or other oils such as primrose oil, borage oil, or soybean oil, whereby the weight ratio of fish oil to other oils most suitably ranges from about 1:50 to about 50:1. For example, the weight ratio of fish oil to primrose oil and/or borage oil, or the ratio of fish oil to soybean oil, may suitably range from about 1:2 to about 1:20. In some embodiments, the mixtures of the EFAs will comprise at least omega 3 and omega 6 fatty acids at a ratios of 1:1-1:40—Physiologically ideal ratio is 1:1.7 so a most preferable range would be 1:1.5-4, with 1:4-8 also being useful.

Suitable fat emulsions of the present invention preferably contain one or more omega-3 fatty acids and/or omega-6 fatty acids and/or their pharmaceutically tolerable ester or salts present in quantities ranging from about 5 to about 45% by weight, preferably in quantities ranging from about 10% to about 30% by weight, and most preferably in quantities ranging from about 10% to about 20% by weight. Useful mixtures include, but are not limited to dilution of 10% and 20% by weight mixtures.

For suitable fat emulsions containing one or more omega-3 fatty acids, the fatty acids, their esters or salts in pure form or in the form of components of oils are preferred for use in accordance with the present invention.

Fat emulsions of the present invention may also include one or more physiologically safe emulsifiers. Suitable emulsifiers include for example but are not limited to phospholipids with an animal or vegetable origin, and preferably those phospholipids which contain EPA as a polyunsaturated fatty acid. Ovolecithin is particularly suitable for use in compositions of the present invention. Other useful emulsifiers include synthetic and semi-synthetic lecithins. Such one or more emulsifiers may be present in the subject fat emulsion in quantities ranging from about 1% to about 20% by weight (based on the fat content), and preferably in quantities ranging from about 5% to about 15% by weight (based on the fat content).

The compositions may also contain other biologically active compounds such as antioxidants or agents known to scavange or counteract the effects of toxic free radicals and byproducts of oxidative and other chemical manifestations of physiologic stress. These include but are not limited to Vitamin E, Vitamin C, Caratenoids, flavonoids, Lipoic acid any derivatives thereof or mixtures. Vitamin E, natural, synthetic, mixed tocopherols. Vitamin E, is preferably in the form of tocopherol or a pharmaceutically safe tocopherol ester, such as for example but not limited to tocopherol acetate, may be used in the subject fat emulsion in quantities ranging from about 0.15% to about 1.5% by weight (based on the fat content), to act as an antioxidant. Other compounds can be present

Additional suitable additives may be included in the subject fat emulsion such as for example but not limited to conventional isotonic additives (common intravenous salts such as sodium chloride and nonelectrolytes such as glucose, pH modifiers(such as acetic acid and sodium acetate) and buffers (such as acetate and phosphate buffer systems composed of the acid and a salt of the acid), emulsion stabilizers like gelatin, long chain sugars like agar and/or co-emulsifiers like tweens and spans, as well as selenium compounds, if desired. It is common to poise intravenous products to an osmolarity of approximately 300 milliosmols/liter and a pH of approximately 7.4, this can be accomplished by the use of tonicity adjusters and buffers by one skilled in the preparation of medications that are to be delivered to the patient via the intravenous route of administration.

Suitable isotonic additives include for example but are not limited to the commonly employed isotonic agents glycerin, glucose, xylose, and sorbite, with glycerin being preferred.

For purposes of illustration and not limitation, formulations of two suitable fat emulsions for use in compositions of the present invention are set forth below in Table 1 and Table 2.

TABLE 1
Fat Emulsion Formulation

| | | |
|---|---|---|
| Fish oil | 100 | mg/ml |
| Glycerin (isotonic agent) | 25 | mg/ml |
| Ovolecithin | 12 | mg/ml |
| Vitamin E | 0.15 | mg/ml |

| | |
|---|---|
| Water (for injection) | to make 1 ml |

TABLE 2
Fat Emulsion Formulation

| | | |
|---|---|---|
| EPA/DHA | 75 | mg/ml |
| Glycerin (isotonic agent) | 25 | mg/ml |
| Ovolecithin | 12 | mg/ml |
| Vitamin E | 0.15 | mg/ml |

| | |
|---|---|
| Water (for injection) | to make 1 ml |

The fish oil used in the formulation of Table 1 above is preferably highly refined fish oil that has been enriched in omega-3 fatty acids as triglyceride components by means of techniques known to those skilled in the art such as that disclosed in DE PS 37 22 540. Such preferred fish oil contains at least about 40% by weight omega-3 fatty acids. The total EPA and DHA content of the fish oil as triglyceride components ranges from about 25% to about 50% by weight, and more preferably ranges from about 35% to about 50% by weight (each value determined on the basis of the surface percentage in a gas chromatogram). The EPA and DHA content of the fish oil can be present in varying quantitative ratios, which can be determined by measuring the respective surfaces in the gas chromatogram. The quantitative ratios depend on the nature of the fish oil used, and on the degree of enrichment of omega-3 fatty acids achieved. Fish oils in which EPA and DHA as triglyceride components are present in a quantitative ratio of EPA to DHA from about 0.5:1 to about 2.6:1 (surface ratio in the gas chromatogram), are preferred for the subject fat emulsions.

Fat emulsions used in accordance with the invention are oil-in-water emulsions (O/W) for which the external phase consists of distilled water, suitable for intravenous administration. Intravenous administration of compositions of the present invention including an effective amount of one or more EFAs or fat emulsions including one or more EFAs such as one or more polyunsaturated, long-chain omega-3 fatty acids, omega-6 fatty acids or their pharmaceutically tolerable esters or salts through a vascular access prior to or during dialysis significantly reduces associated complications.

In one embodiment, compositions of the present inventions including one or more EFAs or a fat emulsion of one or more EFAs, is intraveneously administered during hemodialysis. In such a case, the composition is administered by Intradialysis Infusion of 10% Fish Oil Emulsion.

Materials and Methods

The preparation for infusion during dialysis is a fish oil emulsion containing 10 g to 20 g of fish oil, 2.5 g glycerol and 1.2 g egg-yolk lecithin per 100 ml (Omegaven®, Fresenius Kabi, Bad Homburg, Germany) making up a 10 to 20% solution. The fish oil is highly refined and contains at least 40% long chain omega-3 fatty acids (EPA, DHA) along with other long chain saturated and unsaturated fatty acids. The omega-3:omega-6 ratio can be selected from 1:2 to 1:4 depending on the combination of 10-20% fat from ®® and 80-90% from a basic long chain emulsions of soybean oil.

Omegaven® is available as a commercial product in sterile glass vials containing 50 or 100 ml of the 10% emulsion. The vial should be checked for any precipitation and discarded if present. The container should be shaken before use and the contents accessed only via sterile procedure and infusion sets. The contents can only be used for infusion via a central or peripheral vein or through a dialysis machine. The emulsion is infused into the drip chamber for the venous blood line at the distal end of the dialyzer.

The infusion of Omegaven® should only begin after approximately 15 minutes of dialysis and be infused continuously at a rate not to exceed 0.5 ml/kg/hour so as to avoid hypertrigleceridemia noted with more rapid infusion. A 20% omega-3 concentration can be infused over 2.5 to 3 hours if the 10% cannot meet the dosage requirements of approximately 4 g per dialysis session.

Omegaven® can be infused with other emulsions or solutions providing there is no incompatability as per manufacturer's instructions, and is preferably administered through the same vascular access port during the dialysis. The rate of administration can vary, but will general result in a a total dosage of composition of 25 g to 25 g during the dialysis procedure.

In an alternative embodiment, compositions of the present invention may be administered prior to, or in preparation for, hemodialysis. In such cases the composition is administered intravenously at preferably the same dosage as above.

In a typical patient, hemodialysis is administered three times per week. Administration of the subject compositions prior to or during each hemodialysis session is preferred, with administration during hemodialysis being the most preferred. Less frequent administration may be acceptable depending on factors particular to the patient. Such factors include condition of patient's omega-3 fatty acid status as measured by omega- 3 fatty acid content in biologic tissues like red blood cell membranes, platelets, and the like utilizing currently validated measurements like the omega-3 index (measure of the amount of EPA+DHA in Red Blood Cell membranes expressed as the percent of total fatty acids) as well as other conventional as well as emerging measurement technologies that would give the administering physician information about the dose or interval of administration of the present invention to achieve maximal clinical benefits. Some examples of measurements that can be performed to assist in the adjustment of the said medical regimen comprising this invention include but by no means is limited to blood chemistry evaluation for triglycerides, cholesterol, fatty acids and lipoproteins and apoproteins, coagulation studies and markers of coagulability, hepatic, renal and electrolytes, cytokines, membrane and tissue phospholipids, eicosanoids such as prostaglandinss E2, E3, leukotrienes B4 and B5, to name a few, immune markers, markers of endothelial function such as eNO synthase activity, nitric oxide, glutamine and other intermediaries, adhesion molecules, by products of oxidative stress, free radicals and surrugate markers for lipooxidation, superoxide production, markers of inflammation like c-reactive protein as well as markers of autoimmunity, cell proliferation and any measurable marker or byproduct of metabolic processes that will enable the prescribing physician to determine whether the dose of the current invention is meeting, its goal in its present dose or dosing interval regimen. Other physical findings such as blood pressure, heart rate may also be used to adjust dose, as well as tests such as Flow Mediated Dilatation for endothelial function. In a preferred embodiment, a composition of the present invention comprising an effective amount of approximately 4 grams of one or more essential fatty acids or approximately 4 grams of one or more essential fatty acids in a fat emulsion is administered per dialysis session.

The present invention also provides for determining, adjusting, or optimizing the dosage of the compositions for individual patients based on each patient's physical and physiological condition and status. Factors that may influence the dosage include, for example, age, weight, body mass index, body surface area, gender, racial or ethnic background, personal and family medical history, preexisting illnesses or conditions, risk factors for diseases or conditions, and the result of lab work. Based on consideration of one or more such factors a starting dose may be determined, and the dose adjusted on a periodic basis. For example, patients with hypertriglyceridemia (TGs>250 mg) should be started on a lower dose or slower rate of infuson. Grounds for downward adjustment of the dose may include development of hypertriglyceridemia above 250 mg when measured within 90 minutes of initiating infusion, while those for upward adjustment of the dose may include inadequate rise in desired omega-3 fatty acid levels in target tissues or the unsatisfactory suppression of inflammatory markers or metabolic intermediates known to be surrogate markers for achieving the clinical benefit of the invention. Further, it is desirable to monitor the blood chemistry of each patient to determine whether the dosages should be modified. Parameters which can be monitored can include triglyceride levels. The dose adjustments that may be based on the results of such monitoring can include. Such blood chemistry measurements can be made on a periodic basis, such as every 3-6 months, or preferably every 1 to 3 months, but may also include measurements within the first 24 hours to 30 days of administration. After a dose adjustment is made, it is desirable to allow a period of time for the patient's condition to equilibrate or stabilize before determining if the adjusted dose should be continued or further modified. A desirable period of time to wait in order to evaluate the result of an adjustment is 3 months, but other periods may be utilized as circumstances dictate.

Compositions of the present invention comprise EFAs or EFAs in a fat emulsion alone or alternatively in combination with one or more active pharmaceutical ingredients and/or nutritional supplements. Suitable nutritional supplements include for example but are not limited to ALA, B group vitamins, B group vitamin derivatives, vitamin E, vitamin D, vitamin A, Caretenoids, alpha lipoic acid, flavenoids, vitamin K, statins, fibric acid derivatives, iron, erythropoeitin, CoQ10, amino acids, creatin, camitine, zinc, calcium, PTH, PTH analogs, chelators, lipids, proteins, carbohydrates and combinations thereof. Such compositions, when present, can be in forms which can be utilized phiologically.

Examples of such agents also include: neuroprotectants such as nimodipine and related compounds; antibiotics such as tetracycline, chlortetracycline, bacitracin, neomycin, polymyxin, gramicidin, oxytetracycline, chloramphenicol, gentamycin, and erythromycin; antiinfectives; antibacterials such as sulfonamides, sulfacetamide, sulfamethizole, sulfisoxazole; nitro furazone, and sodium propionate; antiallergenics such as antazoline, methapyriline, chlorpheniramine, pyrilamine and prophenpyridamine; bacterostatic or microbiostatic agents or preservatives, anti-inflammatories such as hydrocortisone, hydrocortisone acetate, dexamethasone 21-phosphate, fluocinolone, medrysone, methyiprednisolone, prednisolone 21-phosphate, prednisolone acetate, fluoromethalone, betamethasone and triminolone; miotics and anti-cholinesterase such as pilocarpine, eserine salicylate, carbachol, di-isopropyl fluorophosphate, phospholine iodine, and demecarium bromide; mydriatics such as atropine sulfate, cyclopentolate, homatropine, scopolamine, tropicamide, eucatropine, and hydroxyamphetamine; sympathomimetics such as epinephrine; and prodrugs such as thjose described in Design of Prodrugs, edited by Hans Bundgaard, Elsevier Scientific Publishing Company, Amerstdam, 1985, incorporated herein by reference. In addition to the above agents, other agents suitable for intravenously treating, managing, or diagnosing conditions in a mammalian organism may be added to compositions of the present invention provided there is no incompatibility with the other components of the composition. Reference may be made to any standard pharmaceutical textbook such as Remington's Pharmaceutical Sciences for the identity of such agents.

Because the formulations are to be introduced intravenously, they must, by necessity, be sterile, and preferably contain preservatives to maintain sterility. Two classes of preservatives that have particular utility with emulsions of essential fatty acids are salts of edetate (ethylenediaminetetraacetic acid) and pedetate (diethylenetriaminepentaacetic acid). Generally, for edetate preferred salts include sodium and calcium edetate, with disodium edetate being preferred. For pedetate, preferred salts will exhibit less affinity for the pedetate than calcium, with calciumtrisodium pedetate being preferred. Both salts are preferably present at low concentrations, with edetate present at 0.03-0.9 millimolar and pedetate at 0.0005-0.005% by weight. Generally, an effective preservative fulfils the function of preventing significant growth of microorganisms for at least 24 hours in the event of adventitious extrinsic contamination (e.g. preferably no more than 10-fold increase following a low level of extrinsic contamination, such as 10-10000 colony forming units, at temperatures in the range of 20.degree.-25.degree. C.). In useful assay, broth cultures of one or more standard USP (United States Pharmacopeia) preservative efficacy test organisms are added to preservative containing formulations at approximately 100-200 colony forming units per ml. The test formulations were incubated at 25-30.degree. C. and tested for viable counts after 24 and 48 hours.

Intravenous administration of compositions of the present invention without the addition of one or more active pharmaceutical agents, may be further beneficial to the patient for indications including hypertension, cardiovascular risk reduction, nutritional supplementation, inflammation modulation, immunomodulation, neuropsychiatric modulation, acute illness, arrhythmias and malignancies.

Compositions of the present invention may be produced using commercially available EFAs or EFA emulsions suitable for intravenous administration. One such EFA emulsion is Omegaven®, produced by Fresenius Kabi, Bad Homburg, Germany. The qualitative and quantitative composition of 100 ml Omegaven(g) emulsion contains: 10.0 g highly refined fish oil containing: eicosapentaenoic acid (EPA) 1.25-2.82 g; docosahexaenoic acid (DMA) 1.44-3.09 g; myristic acid 0.1-0.6 g; palmitic acid 0.25-1.0 g; palmitoleic acid 0.3-0.9 g; stearic acid 0.05-0.2 g; oleic acid 0.6- 1.3 g; linoleicacid 0.1-0.7 g; linolenic acid 0.2 g; octadecatetraenoic acid 0.05-0.4 g; eicosaenoic acid 0.05-0.3 g; arachidonic acid 0.1-0.4 g; docosaenoic acid 0.15 g; docosapentaenoic acid 0.15-0.45 g; dl-a-Tocopherol (as an antioxidant) 0.015-0.0296 g; Glycerol 2.5 g; Purified egg phosphatide 1.2 g; Total energy: 470 kJ/100 ml=112 kcal/100 ml. pH value: 7.5 to 8.7. Titration acidity: <1 mmol HCl/I. Osmolality: 308-376 mosm/kg. The pharmaceutical form is an emulsion for infusion. Therapeutic indications include parenteral nutrition supplementation with long chain omega-3 fatty acids, especially eicosapentaenoic and docosahexaenoic acid, when oral or enteral nutrition is impossible, insufficient or contraindicated. The maximum infusion rate should not exceed 0.5 ml Omegaven®/kg body weight/hour corresponding to 0.05 g fish oil/kg body weight/hour.

An embodiment of the present invention for illustration not limitation, is a method of preparing a composition of the present invention comprising combining a fish oil emulsion containing 10 g to 20 g of fish oil, 2.5 g glycerol and 1.2 g egg-yolk lecithin per 100 ml (Omegaven®), making up a 10% to 20% solution. The fish oil is highly refined and contains at least 40% long chain omega-3 fatty acids. The omega-3: omega-6 ratio can be selected from 1:2 to 1:4 depending on the combination of 10-20% fat from Omegaven® and 80-90% fat from basic long chain emulsions of soybean oil. A method of using the prepared composition comprises intravenously administering the composition containing an effective amount of an EFA emulsion to a patient prior to or during hemodialysis at a rate not to exceed 0.5 ml/kg/hour so as to avoid hypertrigleceridemia noted with more rapid infusion. for a total dosage of 4 gram of omega-3 fatty acids per dialysis session. A 20% omega-3 concentration can be infused over 2.5 to 3 hours if the 10% cannot meet the dosage requirements determined by the target clinical and biochemical goals measured from time to time.

Omegaven® is available as a commercial product in sterile glass vials containing 50 or 100 ml of a 10% emulsion. The vial should be checked for any precipitation and discarded if precipitation is present. The container should be shaken before use and the contents accessed only via sterile procedure and infusion sets. Omegaven® can only be used for infusion via a central or peripheral vein or through a dialysis machine. The emulsion may also be infused into the drip chamber for the venous blood line at the distal end of the dialyzer.

The infusion of Omegaven® should only begin after approximately 15 minutes of dialysis and infused continuously at a rate not to exceed 0.5 ml/kg/hour so as to avoid hypertrigleceridemia noted with more rapid infusion. A 20% omega-3 concentration can be infused over 2.5 to 3 hours if the 10% cannot meet the dosage requirements of approximately 4 g per dialysis session.

Omegaven® can be infused with other emulsions or solutions providing there is no incompatibility as per manufacturer's instructions.

### EXAMPLES

### Example 1:

A fish oil emulsion is prepared for intravenous administration during hemodialysis. The fish oil emulsion contains 10 g of fish oil, 2.5 g glycerol and 1.2 g egg-yolk lecithin per 100 ml, i.e., Omegaven® (Fresenius Kabi, Bad Homburg, Germany), making up a 10% solution. The fish oil is highly refined and contains at least 40% long chain omega-3 fatty acids. The omega-3: omega-6 ratio is 1:4.

The commercially available sterile glass vial of Omegaven® is checked for any precipitation and discarded if present. The container is thoroughly shaken and the emulsion container therein is accessed via an infusion set using standard sterile procedures. The emulsion is infused into the drip chamber for the venous blood line at the distal end of the dialyzer.

The infusion of the Omegaven® emulsion begins after approximately 15 minutes of dialysis. The emulsion is infused continuously at a rate not to exceed 0.5 ml/kg/hour until 4 g of the emulsion have been infused.

### Example 2

A 20% omega-3 concentration emulsion is infused over 2.5 to 3 hours, according to Example 1, to meet the dosage requirements of approximately 4 g per dialysis session.

### Example 3

A fish oil and vegetable oil emulsion combination is prepared for intravenous administration during hemodialysis. The combination emulsion contains 10 g of fish oil, 2.5 g glycerol and 1.2 g egg-yolk lecithin per 100 ml, i.e., Omegaven® (Fresenius Kabi, Bad Homburg, Germany), in combination with a flax seed oil emulsion containing 5 g of flax seed oil i.e., ALA 75 (BioGin Biochemicals Co., Ltd, Chengdu, China) making up a 15% solution. The fish oil is highly refined and contains at least 40% long chain omega-3 fatty acids with an omega-3: omega-6 ratio of 1:4 and the flax seed oil contains at least 70% long chain omega-3 fatty acids with an omega-3:omega-6 ratio of 4:1 ratio.

The emulsion is infused into the drip chamber for the venous blood line at the distal end of the dialyzer. Alternatively, the infusion can also be administered through a central or peripheral venous line.

The infusion of the emulsion begins after approximately 15 minutes of dialysis. The emulsion is infused continuously at a rate not to exceed 0.5 ml/kg/hour until approximately 4 g of the emulsion have been infused.

### Example 4

An emulsion combination wherein omega-3 fatty acids from marine and vegetable sources are combined with high dose folic acid (10 mg) and Vitamin B12 ( 10 mcg) is prepared for intravenous administration during hemodialysis. The combination emulsion contains 10 g of fish oil, 2.5 g glycerol and 1.2 g egg-yolk lecithin per 100 ml, i.e., Omegaven® (Fresenius Kabi, Bad Homburg, Germany), in combination with a flax seed oil emulsion containing 5 g of flax seed oil i.e., ALA 75 (BioGin Biochemicals Co., Ltd, Chengdu, China) making up a 15% solution. The fish oil is highly refined and contains at least 40% long chain omega-3 fatty acids with an omega-3: omega-6 ratio of 1:4 and the flax seed oil contains at least 70% long chain omega-3 fatty acids with an omega-3:omega-6 ratio of 4:1 ratio. The folic acid is in the form of 5-FornylH₄folate (folinic acid) and is administered clinically under the name Leucovorin™ Leucovorin Calcium 10 mg/ml Intravenous Injection Solution.

The emulsion is infused into the drip chamber for the venous blood line at the distal end of the dialyzer or via a central port or peripheral venous line.

The infusion of the emulsion begins after approximately 15 minutes of dialysis. The emulsion is infused continuously at a rate not to exceed 0.5 ml/kg/hour until approximately 4 g of the omega-3 fatty acids have been infused and the 10 mg of folinic acid. Based on measurements of endothelial function like Flow Mediated Dilatation (FMD), the folinic acid could be increased or decreased to achieve the desired clinical outcome. Additional examples, either paper or based on experimental results showing: other formulations, other routes of administration, use of blood chemistry monitoring to determine proper dosage, various optimized dosages, etc.

Having described the invention in detail, those skilled in the art will appreciate that modifications may be made of the invention without departing from its spirit and scope. Therefore, it is not intended that the scope of the invention be limited to the specific embodiments described. Rather, it is intended that the appended claims and their equivalents determine the scope of the invention.

It is apparent that many modifications and variations of the invention as hereinabove set forth may be made without departing from the spirit and scope thereof. The specific embodiments described are given by way of example only, and the invention is limited only by the terms of the appended claims.

We claim:

1. A composition comprising:
an effective amount of pure form essential fatty acids or a fat emulsion of said pure form essential fatty acids suitable for intravenous administration prior to or during hemodialysis.

2. The composition of claim 1 wherein said pure form essential fatty acids are selected from the group consisting of omega-3 fatty acids, salts of omega-3 fatty acids, esters of omega-3 fatty acids, omega-6 fatty acids, salts of omega-6 fatty acids, esters of omega-6 fatty acids and combinations thereof.

3. The composition of claim 1 wherein said fat emulsion is an oil-in-water emulsion.

4. The composition of claim 1 wherein said pure form essential fatty acids include eicosapentaenoic acid and docosahexaenoic acid.

5. The composition of claim 1 wherein said pure form essential fatty acids include eicosapentaenoic acid and docosahexaenoic acid in a ratio of eicosapentaenoic acid to docosahexaenoic of about 0.5:1 to about 2.6:1.

6. The composition of claim 1 wherein the composition is suitable for intravenous administration three times per week.

7. The composition of claim 1 wherein said effective amount is about 4 grams.

8. The composition of claim 1 further comprising a component selected from the group consisting of active pharmaceutical ingredients, nutritional supplements, and mixtures thereof.

9. The composition of claim 1 further comprising a component selected from the group consisting of B group vitamins, B group vitamin derivatives, vitamin E, vitamin D, vitamin A, vitamin K, statins, fibric acid derivatives, iron, erythropoeitin, CoQ20, lutein, creatin, camitine, zinc, calcium, PTH, PTH analogs, chelators, lipids, proteins, carbohydrates and mixtures thereof.

10. The composition of claim 1 wherein the administration of said composition is useful in treating at least one indication selected from the group consisting of hypertension, cardiovascular risk reduction, nutritional supplementation, inflammation modulation, immunomodulation, neuropsychiatric modulation, acute illness, arrhythmias and malignancies.

11. The composition of claim 1 further comprises essential fatty acids in oils.

12. A method of preventing, stabilizing, reversing and/or treating of one or more complications associated with a vascular access comprising:
intravenously administering a composition including essential fatty acids or a fat emulsion of essential fatty acids through the vascular access.

13. The method of claim 12 wherein the vascular access is utilized for hemodialysis.

14. The method of claim 12 wherein the composition is administered during hemodialysis.

15. The method of claim 12 wherein the composition is administered prior to hemodialysis.

16. The method of claim 12 wherein the composition is administered three times per week.

17. The method of claim 12 wherein about 4 grams of essential fatty acid is administered.

18. The method of claim 12 wherein the composition is administered in combination with a component selected from the group consisting of active pharmaceutical ingredients, nutritional supplements, and mixtures thereof.

19. The method of claim 12 wherein the composition is administered in combination with at least one additive selected from the group consisting of B group vitamins, B group vitamin derivatives, vitamin E, vitamin D, vitamin A, vitamin K, statins, fibric acid derivatives, iron, erythropoeitin, CoQ10, lutein, creatin, camitine, zinc, calcium, PTH, PTH analogs, chelators, lipids, proteins, carbohydrates and mixtures thereof.

20. The method of claim 12 wherein the administration of the composition is useful for at least one indication selected from the group consisting of hypertension, cardiovascular risk reduction, nutritional supplementation, inflammation modulation, immunomodulation, neuropsychiatric modulation, acute illness, arrhythmias and malignancies.

21. The method of claim 12 wherein the composition is administered to a patient not having renal disease.

22. The method of claim 12 wherein the composition includes Omegaven®.

23. A method for preventing stenosis or thrombosis of a hemodialysis patient's vascular access comprising:
administering an emulsion of essential fatty acids through the vascular access.

24. The method of claim 23 wherein the emulsion includes Omegaven®.

25. The method of claim 23 wherein the emulsion is administered at a rate of no more than about 0.5 ml/kg/hour.

26. The method of claim 23 wherein the emulsion is administered until about 4 g of the emulsion has been administered to the patient.

27. The method of claim 23 wherein the emulsion is administered in combination with a component selected from the group consisting of active pharmaceutical ingredients, nutritional supplements, and mixtures thereof.

28. The method of claim 23 wherein the emulsion is administered in combination with at least one additive selected from the group consisting of B group vitamins, B group vitamin derivatives, vitamin E, vitamin D, vitamin A, vitamin K, statins, fibric acid derivatives, iron, erythropoeitin, CoQ10, lutein, creatin, camitine, zinc, calcium, PTH, PTH analogs, chelators, lipids, proteins, carbohydrates and mixtures thereof.

29. The method of claim 23 wherein the administration of an essential fatty acid is further indicated by at least one of the indications selected from the group consisting of hypertension, cardiovascular risk reduction, nutritional supplementation, inflammation modulation, immunomodulation, neuropsychiatric modulation, acute illness, arrhythmias and malignancies.

30. A method of preventing, stabilizing, reversing and/or treating of one or more complications associated with a vascular access in a patient, as well as preventing complications from administration of essential fatty acids, comprising:
intravenously administering a predetermined dose of composition including essential fatty acids or a fat emulsion of essential fatty acids through the vascular access to intravenously through another access;
monitoring said patient for a response to said dose; and
adjusting subsequent doses up or down based upon the response observed in said monitoring.

31. The method of claim 30 wherein the initial predetermined dose is 4 grams of essential fatty acid administered to a patient.

32. The method of claim 31 wherein the initial predetermined dose is based upon a medical history taken of said patient.

33. The method of claim 31 wherein the medical history includes one or more factors selected from the group consisting of age, weight, body mass index, body surface area, gender, racial or ethnic background, personal and family medical history, preexisting illnesses or conditions, risk factors for diseases or conditions, and results of lab work, and the initial predetermined initial dose is adjusted up or downward based on said one or more factors.

34. The method of claim 31, wherein said patient is undergoing dialysis and the predetermined initial dose is given to a patient at a frequency of prior to, concurrent with, or after each dialysis session.

35. The method of claim 34 wherein the frequency and/or initial predetermined initial dose is varied based upon a medical history taken of the patient.

36. The method of claim 35 wherein the medical history includes one or more factors selected from the group consisting of age, weight, body mass index, body surface area, gender, racial or ethnic background, personal and family medical history, preexisting illnesses or conditions, risk factors for diseases or conditions, and results of lab work, and the frequency and/or initial predetermined initial dose is adjusted up or downward based on said one or more factors.

37. The method of claim 30 wherein the monitoring of said patient includes monitoring of blood chemistry.

38. The method of claim 38 wherein the monitoring includes monitoring of triglyceride levels.

39. The method of claim 37 wherein the dose or frequency of administration is adjusted up or down based on the results of the blood chemistry monitoring.

40. The method of claim 37, wherein said monitoring is conducted periodically before, during, or after the administration of the essential fatty acid compositions.

41. The method of claim 37 wherein the monitoring occurs over a schedule based on patient condition or monitoring results.

42. The method of claim 41 wherein the monitoring occurs after said patient has had time to equilibrate after a dose or frequency has been changed.
